SEQUENCE LISTING (6 sequences)

SEQ ID NO: 1 - DNA, 20 bp, Artificial Sequence
  Description of Artificial Sequence Synthetic primer
gtacacgcacgctggttacc

SEQ ID NO: 2 - DNA, 20 bp, Artificial Sequence
  Description of Artificial Sequence Synthetic primer
gtagaaagcctcgacatcgc

SEQ ID NO: 3 - DNA, 26 bp, Artificial Sequence
  Description of Artificial Sequence Synthetic primer
gttagggagttgttcggtcataagct

SEQ ID NO: 4 - DNA, 23 bp, Artificial Sequence
  Description of Artificial Sequence Synthetic primer
gccaaggcatatttgccgcggac

SEQ ID NO: 5 - DNA, 24 bp, Artificial Sequence
  Description of Artificial Sequence Synthetic primer
atcgcggcttgttgtttgtctaat

SEQ ID NO: 6 - DNA, 24 bp, Artificial Sequence
  Description of Artificial Sequence Synthetic primer
gggcgaaatgtaggatataaagga

(19) United States
(12) Patent Application Publication
(10) Pub. No.: US 2007/0207453 A1
(43) Pub. Date: Sep. 6, 2007

(54) Multiplex PCR assay

(76) Inventors: Vishali Gupta, Chandigarh (IN); Naresh Sachdeva, Miami, FL (US); Amod Gupta, Chandigarh (IN); Sunil Arora, Chandigarh (IN); Pradeep Bambery, Chandigarh (IN)

Correspondence Address: VENABLE LLP, P.O. BOX 34385, WASHINGTON, DC, 20043-9998, US

(21) Appl. No.: 11/598,094

(22) Filed: Nov. 13, 2006

(30) Foreign Application Priority Data
Nov. 14, 2005 (IN) DEL/3046/2005

(51) Int. Cl. C12Q 1/70; C12Q 1/68; C12P 19/34

(52) U.S. Cl. 435/5; 435/6; 435/91.2

(57) ABSTRACT

This invention relates to a multiplex PCR assay capable of screening or detecting the relevant microbial organism specific to Cytomegalo virus (CMV), Herpes Simplex virus (HSV) and Varicella zoster virus (VZV) present in a sample, comprising a reaction mixture of a combination of three sets of primers, one of said primer set for detection of CMV, a second of said primer sets for detection of HSV, a third primer set for the detection of VZV, said primers being compatible to each other.

### FIELD OF INVENTION

This invention relates to multiplex PCR assay capable of identifying a particular microbial organism in a sample.

### BACKGROUND OF THE INVENTION

Monoplex PCRs is a molecular technique for amplification of a selected gene fragment of the genome of any organism or cell using a specific set of primers specifically designed for that purpose. These primers can recognize and anneal (bind) to their pre-determined (complimentary) sequence on the genome of that cell/organism. Then the reagents including the enzyme, buffers and the nucleotide mix (building blocks) are mixed together in proportion and put at temperature and conditions so that the enzyme can put the building blocks (nucleotides) in pre-specified sequence as per the complementarities to template (parent) strand of DNA.

Such monoplex PCR for diagnosing infections like Cytomegalo virus (CMV), Herpes simplex virus (HSV), Vericella zoster virus (VZV), Human Immunodeficiency virus (HIV), Toxoplasmosis gondii, Mycobacterium tuberculosis, Lyme disease and diseases like lymphomas and Whipple disease have been established. Thus, Monoplex PCR for any infection is known in the art. However, one major impediment to this technique is that one needs to perform a separate PCR reaction for each pathogen that could be time consuming and prohibitively expensive especially if one needs to test for a large number of potential pathogens. Also the monoplex examination would tax the available sample volume that might be very small in situation like intraocular samples.

Multiplex PCR assay is capable of screening various microbial organisms simultaneously or identify different alleles of one organism. In a multiplex PCR, a different cocktail of reagents is used for carrying all other ingredients of a reaction mix as in monoplex PCR situation in addition to the specifically designed primers for all the organisms which are to be identified or detected. However, in Multiplex PCR, the primers and the conditions that are applicable in a monoplex setting no longer produce [[ me]] same results because the primers for different organisms interfere with each other and reduce the sensitivity as well as specificity of assay. Thus both primer selection as well as optimization of conditions and concentration of reagents used need to be standardized, keeping in view the ‘nature’ of each and every primer as well as requirement of the sensitivity in that particular situation.

Dabil and coworkers have published earlier a technique of multiplex PCR assay, by using novel set of primers for a panel of common pathogens including CMV, HSV, VZV and Toxoplasma gondii ( Ref: Dabil H, Boley M L, Schmitz T M and Van Gender R N. Validation of a diagnostic multiplex polymerase chain reaction assay for infectious posterior uveitis. Archives of Ophthalmol 2001; 119:1315-22). Persson and Oslen have published Multiplex PCR reaction for identification of Campylobacter Coli and Campylobacter jejuni from pure cultures and directly on stool samples Persson S and Oslen K E. J Med Microbiol. 2005; 54:1043-7).

### OBJECTS OF THE INVENTION

An object of this invention is to propose a multiplex PCR assay capable of identifying the relevant microbial organism present in a sample.

Another object of this invention is to propose primers for detection of cytomegalo virus (CMV), Herpes Simplex virus (HSV) and Varicella zoster virus (VZV) present in a sample.

Further object of this invention is to propose a reaction mixture which can detect two or three organism in a sample at a time.

### SUMMARY OF THE INVENTION

According to this invention there is provided a multiplex PCR assay capable of screening or detecting the relevant microbial organism specific to Cytomegalo virus (CMV), Herpes Simplex virus (HSV) and Varicella zoster virus (VZV) present in a sample comprising a reaction mixture of a combination of three sets of primers, one of said primer set for detection of CMV, a second of said primer sets for detection of HSV, a third primer set for the detection of VZV, said primers being compatible to each other.

### DETAILED DESCRIPTION OF THE INVENTION

The present invention relates to multiplex PCR reactions for a triplex for CMV, HSV and VZV.

Primer for CMV having the following sequence:

### For Cytomegalo Virus (CMV)

| | | |
|---|---|---|
| ACMVF: | GTA CAC GCA CGC TGG TTA CC | (SEQ ID NO:1) |
| ACMVR: | GTA GAA AGC CTC GAC ATC GC | (SEQ ID NO:2) |

### For Herpes Simplex Virus (HSV)

| | |
|---|---|
| HSV-1F: | |
| GTT AGG GAG TTG TTC GGT CAT AAG CT | (SEQ ID NO:3) |
| HSV-1RA: | |
| GCC AAG GCA TAT TTG CCG CGG AC | (SEQ ID NO:4) |

### For Varicella zoster virus (VZV)

| | | |
|---|---|---|
| VZV F: | ATC GCG GCT TGT TGT TTG TCT AAT | (SEQ ID NO:5) |
| VZV R: | GGG CGA AAT GTA GGA TAT AAA GGA | (SEQ ID NO:6) |

### Reaction Mixture (50 μl)

| | |
|---|---|
| 10X Assay Buffer (0.1 M Tris-HCL, PH 8.8, | −5.0 μl |
| 15 mM MgCl₂, 0.5 M KCI and | |
| 1% Triton-X 100) | |
| 25 mM MgCl₂ | −0.85 μl (total 1.9 mM) |
| 10 mM dNTPs (each of A, T, G, C) | −1.25 μl (250 μM) |
| 50 pmoles/μl ACMV-F | −0.4 μl (0.4 pmoles/μl) |
| 50 pmoles/μl ACMV-R | −0.4 μl (0.4 pmoles/μl) |
| 50 pmoles/μl HSV-1F | −0.5 μl (0.5 pmoles/μl) |
| 50 pmoles/μl HSV-1RA | −0.5 μl (0.5 pmoles/μl) |
| 50 pmoles/μl VZV-F | −0.5 μl (0.5 pmoles/μl) |
| 50 pmoles/μl VZV-R | −0.5 μl (0.5 pmoles/μl) |
| 5 U/μl Taq pol | −1.0 μl (5 units) |
| Distilled water | −36.5 μl |
| Template DNA | −1.0 μl of each organism |

### Temperature And Conditions

The thermocycling was carried out for 35 cycles, with denaturation at 94° C. for 35 sec, annealing at 57° C. for 35 sec and extension at 72° C. also for 35 sec with last cycle of extension of 5 mins. The PCR products were visualized on a 2.5% agarose gel.

We claim:

1. A multiplex PCR assay capable of screening or detecting the relevant microbial organism specific to Cytomegalo virus (CMV), Herpes Simplex virus (HSV) and Varicella zoster virus (VZV) present in a sample comprising a reaction mixture of a combination of three sets of primers, one of said primer set for detection of CMV, a second of said primer set for detection of HSV, a third primer set for the detection of VZV, said primers being compatible to each other.

2. The multiplex PCR assay as claimed in claim 1 wherein the primer for CMV is:

| | | |
|---|---|---|
| ACMVF: | GTA CAC GCA CGC TGG TTA CC | (SEQ ID NO:1) |
| ACMVR: | GTA GAA AGC CTC GAC ATC GC | (SEQ ID NO:2) |

HSV is:

| | |
|---|---|
| HSV-1F: | |
| GTT AGG GAG TTG TTC GGT CAT AAG CT | (SEQ ID NO:3) |
| HSV-1RA: | |
| GCC AAG GCA TAT TTG CCG CGG AC | (SEQ ID NO:4) |

VZV is:

| | | |
|---|---|---|
| VZV F: | ATC GCG GCT TGT TGT TTG TCT AAT | (SEQ ID NO:5) |
| VZV R: | GGG CGA AAT GTA GGA TAT AAA GGA | (SEQ ID NO:6) |

3. The multiplex assay as claimed in claim 1 wherein said reaction mixture comprises:—
Reaction Mixture (50 μ):

| | |
|---|---|
| 10X Assay Buffer (0.1 M Tris-HCL, PH 8.8, | −5.0 μl |
| 15 mM MgCl₂, 0.5 M KCI and | |
| 1% Triton-X 100) | |
| 25 mM MgCl₂ | −0.85 μl (total 1.9 mM) |
| 10 mM dNTPs (each of A, T, G, C) | −1.25 μl (250 μM) |
| 50 pmoles/μl ACMV-F | −0.4 μl (0.4 pmoles/μl) |
| 50 pmoles/μl ACMV-R | −0.4 μl (0.4 pmoles/μl) |
| 50 pmoles/μl HSV-1F | −0.5 μl (0.5 pmoles/μl) |
| 50 pmoles/μl HSV-1RA | −0.5 μl (0.5 pmoles/μl) |
| 50 pmoles/μl VZV-F | −0.5 μl (0.5 pmoles/μl) |
| 50 pmoles/μl VZV-R | −0.5 μl (0.5 pmoles/μl) |
| 5 u/μl Taq Pol | −1.0 μl (5 units) |
| Distilled water | −36.5 μl |
| Template DNA | −1.0 μl of each organism |

4. A method for identifying the relevant microbial organism in a sample comprising the steps of
Preparing a mixture of primers compatible to each other;
treating the clinical sample with the said primers after thermo cycling for 35 cycles with denaturation, annealing at 57° C. for 35 sec and extension at 72° C. also for 35 sec with last cycle of extension of 5 mins, detecting the relevant micro-organism after amplification of the specific gene targets on 2.5 agarose gel.

5. A method as claimed in claim 4 wherein the primer for CMV is:

| | | |
|---|---|---|
| ACMVF: | GTA CAC GCA CGC TGG TTA CC | (SEQ ID NO:1) |
| ACMVR: | GTA GAA AGC CTC GAC ATC GC | (SEQ ID NO:2) |

HSV is:

| | |
|---|---|
| HSV-1F: | |
| GTT AGG GAG TTG TTC GGT CAT AAG CT | (SEQ ID NO:3) |
| HSV-1RA: | |
| GCC AAG GCA TAT TTG CCG CGG AC | (SEQ ID NO:4) |

VZV is:

| | | |
|---|---|---|
| VZV F: | ATC GCG GCT TGT TGT TTG TCT AAT | (SEQ ID NO:5) |
| VZV R: | GGG CGA AAT GTA GGA TAT AAA GGA | (SEQ ID NO:6) |

6. A method as claimed in claim 4 wherein said reaction mixture comprises:—
Reaction Mixture (50 μl):

| | |
|---|---|
| 10X Assay Buffer (0.1 M Tris-HCL, PH 8.8, | −5.0 μl |
| 15 mM MgCl₂, 0.5 M KCI and | |
| 1% Triton-X 100) | |
| 25 mM MgCl₂ | −0.85 μl (total 1.9 mM) |
| 10 mM dNTPs (each of A, T, G, C) | −1.25 μl (250 μM) |
| 50 pmoles/μl ACMV-F | −0.4 μl (0.4 pmoles/μl) |
| 50 pmoles/μl ACMV-R | −0.4 μl (0.4 pmoles/μl) |
| 50 pmoles/μl HSV-1F | −0.5 μl (0.5 pmoles/μl) |
| 50 pmoles/μl HSV-1RA | −0.5 μl (0.5 pmoles/μl) |
| 50 pmoles/μl VZV-F | −0.5 μl (0.5 pmoles/μl) |
| 50 pmoles/μl VZV-R | −0.5 μl (0.5 pmoles/μl) |
| 5 U/μl Taq Pol | −1.0 μl (5 units) |
| Distilled water | −36.5 μl |
| Template DNA | −1.0 μl of each organism |
